(19) United States
(12) Patent Application Publication
(10) Pub. No.: US 2020/0368512 A1
(43) Pub. Date: Nov. 26, 2020

(54) MICRONEEDLE DELIVERY SYSTEM WITH ANCHOR

(71) Applicant: EVONIK CORPORATION, PARSIPPANY, NJ (US)

(72) Inventor: Kevin Burton, Hoover, AL (US)

(21) Appl. No.: 16/959,314

(22) PCT Filed: Dec. 31, 2018
(86) PCT No.: PCT/US18/68211

§ 371 Date: Jun. 30, 2020

Related U.S. Application Data
(60) Provisional application No. 62/612,967, filed on Jan. 2, 2018.

(51) Int. Cl. A61M 37/00

(52) U.S. Cl. CPC A61M 37/0015; A61M 2205/0205; A61M 2037/0046; A61M 2037/0023

(57) ABSTRACT

Described herein are microneedle arrays for delivery of medicinal formulations. The microneedle arrays includes an anchoring system for securing the microneedle arrays to the skin for a period of time, e.g., a treatment period. The microneedles in the array also may include a coating, such as a lubricious coating for ease of puncturing/penetrating the skin or anti-microbial agents.

### BACKGROUND OF THE INVENTION

Human skin has three layers: the epidermis, the outermost layer of skin, which provides a waterproof barrier and creates our skin tone; the dermis, which is beneath the epidermis, and contains tough connective tissue, hair follicles, and sweat glands; and the hypodermis, which is a deeper subcutaneous tissue that is made of fat and connective tissue. An outermost layer of the epidermis is the stratum corneum, which functions to form a barrier to protect underlying tissues from infection, dehydration, chemicals and mechanical stresses.

Microneedle arrays are minimally invasive devices that are applied to the skin surface to deliver medicinal formulations through the skin. See, for example, FIG. 1, illustrating the layers of skin and a microneedle array coupled to the skin surface. Microneedles are typically 50-900 mm in height. Microneedles can be arranged in an array of up to 2000 cm² in various geometries and materials (e.g., silicon, metal, polymer) using microfabrication techniques. Microneedles are applied to the skin surface and painlessly pierce the epidermis, creating microscopic channels through which the medicinal formulations diffuse through the epidermis to the dermal microcirculation. Microneedles are long enough to penetrate to the dermis, but are short and narrow enough to avoid stimulation of dermal nerves or puncture of dermal blood vessels.

### SUMMARY OF THE INVENTION

In one embodiment, the invention provides a microneedle array for delivery of medicinal formulations. The microneedle array includes an anchoring system for securing the microneedle array to the skin for a period of time, e.g., a treatment period. One or more of the microneedles can include an anchor to securely position the microneedle array on the skin. The microneedles in the array also may include a coating, such as a lubricious coating for ease of puncturing/penetrating the skin or anti-microbial agents. In other constructions, the microneedles may be fabricated from a bioresorbable metal, such as, for example, magnesium.

Other aspects of the invention will become apparent by consideration of the detailed description and accompanying drawings.

### BRIEF DESCRIPTION OF THE DRAWINGS

FIG. 1 illustrates a cross-sectional view of human skin with a microneedle array applied to the skin.

FIG. 2A schematically illustrates application of a solid microneedle array to human skin.

FIG. 2B schematically illustrates application of a coated microneedle array to human skin.

FIG. 2C schematically illustrates application of a dissolvable/biodegradable microneedle array to human skin.

FIG. 2D schematically illustrates application of a hollow microneedle array to human skin.

FIG. 3 illustrates a microneedle array.

FIG. 4A illustrates an anchoring system for microneedles in a microneedle array according to an embodiment of the present invention.

FIG. 4B illustrates an anchoring system for microneedles in a microneedle array according to an embodiment of the present invention.

FIG. 4C illustrates an anchoring system for microneedles in a microneedle array according to an embodiment of the present invention.

FIG. 4D illustrates an anchoring system for microneedles in a microneedle array according to an embodiment of the present invention.

FIG. 4E illustrates an anchoring system for microneedles in a microneedle array according to an embodiment of the present invention.

FIG. 4F illustrates an anchoring system for microneedles in a microneedle array according to an embodiment of the present invention.

FIG. 4G illustrates an anchoring system for microneedles in a microneedle array according to an embodiment of the present invention.

FIG. 5A illustrates an anchoring system for microneedles in a microneedle array having a swellable portion according to an embodiment of the present invention.

FIG. 5B illustrates the anchoring system of FIG. 5A in an activated state.

FIG. 6A illustrates an anchoring system for microneedles in a microneedle array having a swellable portion according to an embodiment of the present invention.

FIG. 6B illustrates an anchoring system of FIG. 6A in an activated state.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

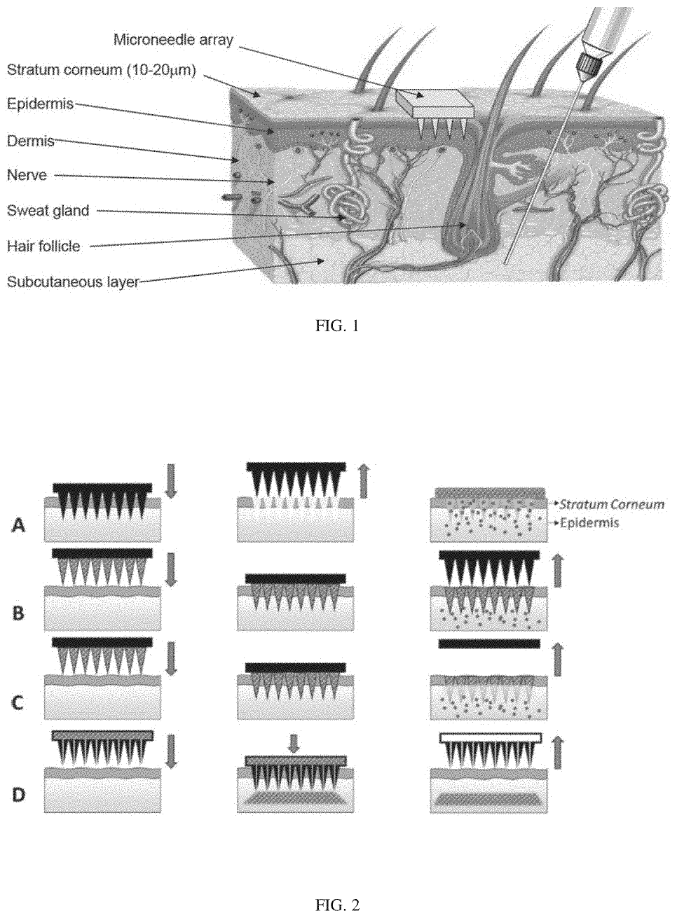

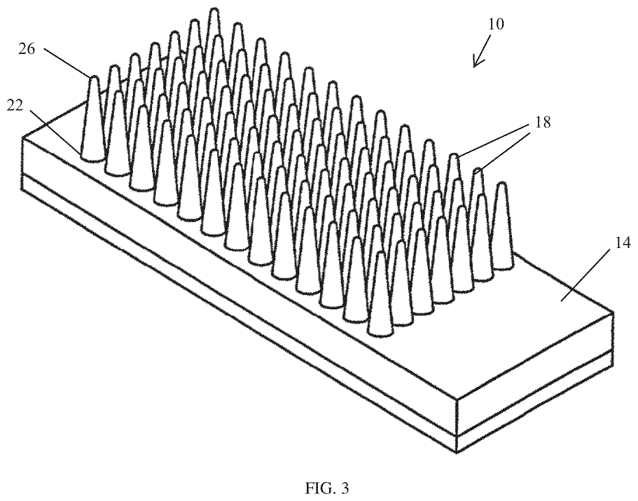

Microneedles can be solid, hollow, or dissolvable/biodegradable. With reference to FIG. 2A, a solid microneedle includes a smooth or seamless outer surface and typically comprises a metal, a polymer, ceramic, semiconductor, material, organic, polymer, composite, silicon, silicon dioxide, bioglass, chitosan, collagen, gelatin, maltose, dextrose, galactose, alginate, agarose, cellulose (such as carboxymethylcellulose or hydroxypropylcellulose), starch, hyaluronic acid, and combinations thereof. Examples of metals include, but are not limited to, dissolvable metals, pharmaceutical grade stainless steel, gold, titanium, nickel, iron, gold, tin, chromium, copper, alloys thereof, and combinations thereof. Examples of polymers may include, but are not limited to, glycolic acid polylactide, polyglycolide, poly lactide, polylactide-co-glycolide, and copolymers with polyethylene glycol (PEG), polyanhydrides, poly(ortho)esters, polyurethanes, poly(butyric acid), poly(valeric acid), poly(lactide-co-caprolactone), and combinations thereof. Examples of non-biodegradable polymers include, but are not limited to, polyethylene glycol, polycarbonate, polymethacrylic acid, ethylenevinyl acetate, polytetrafluorethylene, polyesters, polymers of hydroxy acids such as lactic acid, and combinations thereof. A solid microneedle punctures skin to create temporary microchannels in the epidermis. The solid microneedle is removed from the skin before application of a medicinal formulation (e.g., a patch having a medicinal formulation embedded therein, solution, cream, gel, or other applicator). The medicinal formulation permeates through the microchannels by passive diffusion.

Solid microneedles may be coated with a medicinal formulation prior to insertion into the skin. Coated microneedles (FIG. 2B) are typically prepared by coating a medicinal formulation onto the microneedle outer surface prior to application to the skin. Coated microneedles are employed for the rapid cutaneous delivery of therapeutic agents including small molecules and macromolecules, such as vaccines, proteins, peptides, and DNA to the skin.

A dissolvable/biodegradable microneedle shown in FIG. 2C comprises a material that dissolves or biodegrades while embedded in the skin. A dissolvable/biodegradable microneedle releases its medicinal formulation as the material dissolves or biodegrades in the skin. Dissolvable/biodegradable microneedles are fabricated by micro-moulding soluble matrices, generally a biocompatible polymer or sugar, including the active substance. After insertion of the microneedle into skin, the tip begins to dissolve upon contact with skin interstitial fluid. The medicinal formulation is then released over time. The release kinetics of the medicinal formulation depends upon the constituent polymers' dissolution rate. Therefore, controlled medicinal formulation delivery is achievable by adjusting the polymeric composition of the microneedle, or by modification of the microneedle fabrication process. Dissolvable/biodegradable microneedles provide several advantages. One benefit is the low cost of polymeric materials and their relatively facile fabrication by micromoulding processes at ambient temperatures, which typically allow for straightforward industrial mass production. Various materials, including poly(vinyl alcohol) (PVA), poly(vinylpyrrolidone) (PVP), dextran, carboxymethyl cellulose (CMC), chondroitin sulfate and sugars have all been used to produce this type of microneedle. The use of water-soluble materials eliminates the potential risk of leaving biohazardous sharp waste in the skin. Moreover, safe microneedle disposal is facilitated, since the microneedles are, by definition, self-disabling. One disadvantage of a dissolvable/biodegradable microneedle is the deposition of polymer in skin, possibly making these systems undesirable if they are likely to be used on an ongoing basis.

More specifically, a biodegradable microneedle is produced using biodegradable polymers, including, for example, poly(lactic acid), chitosan, poly(glycolic acid), or poly(lactide-co-glycolide) (PLGA), glycolic acid polylactide, polyglycolide, polylactide-co-glycolide, and copolymers with polyethylene glycol (PEG), polyanhydrides, poly(ortho)esters, polyurethanes, poly(butyric acid), poly(valeric acid), and poly(lactide-co-caprolactone), as well as any of the materials described above with respect to FIG. 2A, to form the matrix. After insertion into skin, the microneedles degrade, rather than dissolve in the skin, releasing their medicinal formulation. Release could possibly be sustained for months by choosing the appropriate polymer. Since biodegradation typically produces small molecules by hydrolysis, polymer is not deposited in the skin indefinitely. However, such microneedles may require high temperatures during manufacture, which may damage biomolecular medicinal formulations.

With reference to FIG. 2D, a hollow microneedle includes a bore formed therein capable of storing a medicinal formulation. After puncture of the skin with a hollow microneedle, the medicinal formulation is released, diffused, or pressure or electrically-driven through the bore from a supply source of the medicinal formulation. A hollow microneedle allows continuous delivery of molecules across the skin through the bore. Hollow microneedles are made from a range of materials, including silicon and metal, glass, polymers, and ceramic. The use of hollow microneedles is limited due to the potential for clogging of the needle openings with tissue during insertion and the flow resistance, due to dense dermal tissue compressed around the tip during insertion.

In some constructions, a microneedle array may include microneedles that all have the same structure. For example, all of the microneedles in the array are solid or all of the microneedles are hollow. In other constructions, a microneedle array may include microneedles having different structures. For example, some of the microneedles in the array are solid and some of the microneedles are hollow. There can be a pattern to the array. For example, the outer microneedles are solid while the inner microneedles are hollow. As another example, a first row comprises solid microneedles, the next adjacent row comprises hollow microneedles, in a continuing alternating pattern. Additional patterns of arrangement of the microneedles in an array are also contemplated.

As noted above, a microneedle array can be utilized for short-term or long-term use depending on the application and the medicinal formulation. For a longer-term use, such as, for example, one week to 6 months, it would be desirable to provide an anchoring system on the microneedles so that the microneedles remain in position during the term of use.

As shown in FIG. 3, a microneedle array 10 includes a base plate 14 and a plurality of microneedles 18 extending from the base plate 14. The base plate 14 may be rigid or flexible depending on the materials used in or the application of the microneedle array 10. The microneedles 18 all extend from the base plate 14 in the same direction. The microneedles 18 each have a base 22 integrally formed with the base plate 14 and a tip 26. The base of the microneedle denotes a proximal end, and the tip denotes a distal end of the microneedle 18. By way of example, the microneedles 18 as illustrated in FIG. 3 have a conical shape where the flat base of the cone is integrally formed with the base plate 14 and tapers smoothly from the flat base to a tip or apex. In other constructions, the shape of the microneedles 18 may be cylindrical (with or without a tapered tip), pyramidal, and the like.

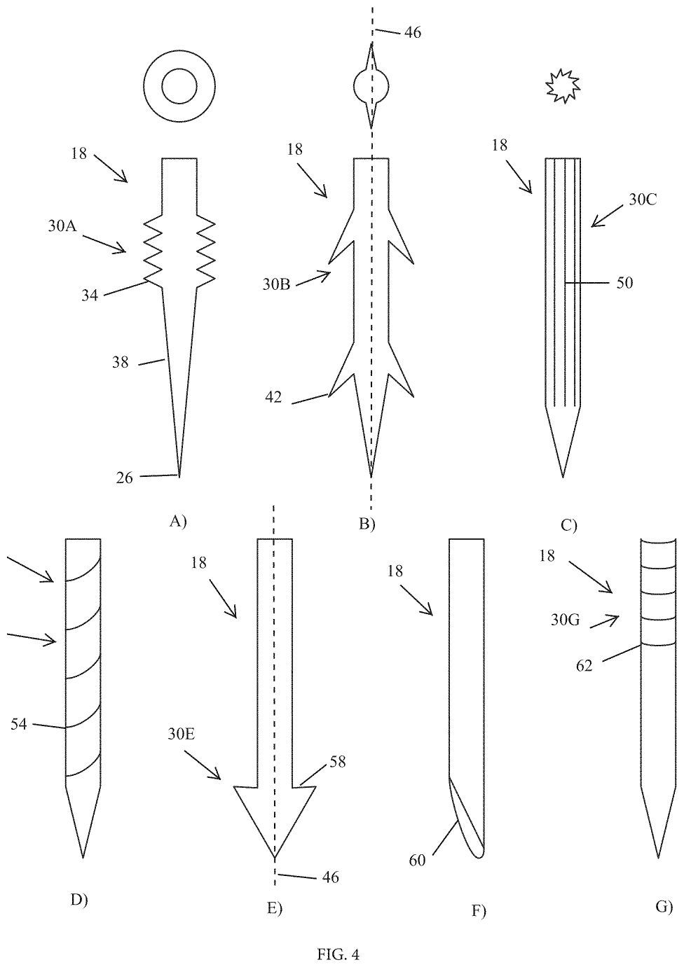

FIGS. 4A-G illustrate an anchoring system 30 integrated with the microneedles 18. FIGS. 4A-G illustrate a single microneedle 18 incorporating the particular anchoring system 30, however it is noted that all or just some of the microneedles 18 in the array 10 can include the particular anchoring system 30.

With reference to FIG. 4A, the anchoring system 30A includes a plurality of protrusions 34 extending radially and circumferentially from an outer surface 38 of the microneedle 18. The protrusions 34 extend perpendicularly (within about 1-2° of a right angle) from the outer surface 38. The protrusions 34 are adjacent to one another meaning there is no visible outer surface between the protrusions 34. The protrusions 34 are positioned in a middle portion of the microneedle 18 and closer to the base 22 than the tip 26. The anchoring system 30A is shown having four protrusions 34, however it is noted that more or fewer protrusions 34 are contemplated.

FIG. 4B illustrates a microneedle 18 having an anchoring system 30B. In this construction, a plurality of barbs 42 extend radially from the outer surface 38 of the microneedle 18. The barbs 42 are oriented at an angle relative to a longitudinal axis 46 of the microneedle 18. A tip of the barb 42 is oriented in the direction of the tip 26 of the microneedle 18. The barbs 42 are positioned at particular locations on the outer surface 38 of the microneedle 18. For example, a first set includes two barbs 42 that are positioned radially opposite one another and toward a distal end of the microneedle 18 while a second set, which includes two barbs are positioned radially opposite one another and toward a proximal end of the microneedle 18. As illustrated, the first set of barbs and the second set of barbs are aligned relative to one another when viewing the cross-sectional drawing, however in other constructions, the second set of barbs may be angularly offset from the first set of barbs.

FIG. 4C illustrates another embodiment of an anchoring system 30C. The anchoring system 30C includes a plurality of grooves 50 formed in the outer surface 38 of the microneedle 18. The grooves 50 are arranged circumferentially around the outer surface 38 with a consistent pattern. The grooves 50 extend between the base 22 and the tip 26 of the microneedle 18. In other constructions of this anchoring system 30C, some or all of the grooves 50 may extend partially along the length of the microneedle 18. In other constructions, the grooves 50 may extend circumferentially around the outer surface 38 in an irregular pattern.

With reference to FIG. 4D, the microneedle 18 shown therein includes an anchoring system 30D including a thread or a tapering groove 54 that spirals toward the tip 26 formed within the outer surface 38. The tapering groove 54, as illustrated extends from the proximal end to the distal end of the microneedle 18. It is noted that the tapering groove 54 may be formed in certain areas of the outer surface 38 and not fully extend from the proximal end to the distal end. It is also noted that the tapering groove 54 may be equally spaced (as illustrated) or may vary in certain areas of the outer surface 38.

FIG. 4E illustrates the microneedle 18 having an anchoring system 30E. The anchoring system 30E includes a radially inward step 58 near the tip 26 of the microneedle 18. The step 58 as illustrated is oriented perpendicularly (within about 1-2° of a right angle) with respect to the longitudinal axis 46, however it is possible that the step 58 may be angularly oriented with respect to the longitudinal axis 46 in other constructions.

FIG. 4F illustrates a further construction of an anchoring system 30F. The anchoring system 30F includes a bevel 60 defining an edge at the tip 26 of the microneedle 18 that assists in securing the microneedle 18 in position. In addition to the bevel 60, the microneedle 18 may include other anchoring systems described herein.

With reference to FIG. 4G, the microneedle 18 includes yet another possible anchoring system 30G. In this construction, the anchoring system 30G includes a plurality of circumferential grooves 62 formed in the outer surface 38 of the microneedle 18. The grooves 62 are positioned at the proximal end of the microneedle 18.

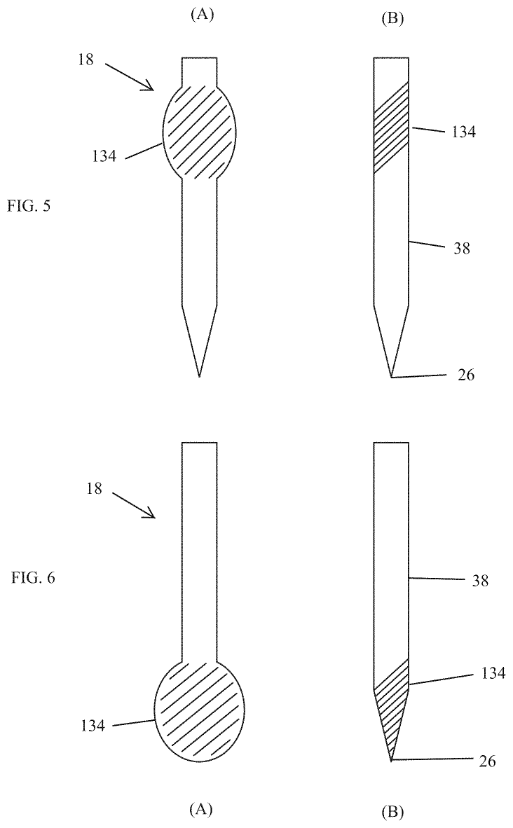

FIGS. 5A-B and FIGS. 6A-B illustrate an anchoring system 130 integrated with the microneedles 18. FIGS. 5A-B and FIGS. 6A-B illustrate a single microneedle 18 incorporating the particular anchoring system 130, however it is noted that all or just some of the microneedles 18 in the array 10 can include the particular anchoring system 130.

The microneedle 18 shown in FIGS. 5A-B and FIGS. 6A-B may be formed of the same materials set forth above with respect to FIG. 2A. In certain locations of the microneedle 18, a swellable portion 134 is formed in the polymer structure of the microneedle 18. The swellable portion 134 also comprises material such as polymethlmethacrylate, polyethylene glycol, hydrogels, superabsorbers, polyvinylpyrrolidone, polyvinyl alcohol, polyacrylic acid, dextran, polysaccharides, cellulosics (CMC, HPMC), hydroxypropyl methacrylamide, xanthan gum, pectins, collagen, gelatin, chitosan, alginate, hyaluronic acid, albumin, starch, polysiloxanylalkyl esters and combinations thereof. The swellable portion 134, in some constructions, may include a polymer that is different than the rest of the material forming the microneedle 18. The swellable portion 134 is capable of changing shape after it comes in contact with water from the skin or other fat from the body after implantation. The materials used for the swellable portion 134 may be selected based on their ability to change when exposed to ambient stimulus, such as temperature, pH, ionic interactions, osmotic pressure or external stimuli such as light, magnetic field, or ultraviolet light.

With reference to FIGS. 5A-B, the swellable portion 134 is positioned closer to the proximal end than the distal end of the microneedle 18. In FIGS. 6A-B, the swellable portion 134 is positioned at the tip 26 or distal end of the microneedle 18. The swellable portion 134 is generally collinear with the outer surface 38 of the microneedle 18 prior to insertion (see FIGS. 5A and 6A) and then expands radially outward to achieve a greater circumference than the outer surface 38 after insertion (see FIGS. 5B and 6B) due to absorption of water, fat or other bodily fluids or other shape-changing property occurs.

The microneedles 18 in the microneedle array 10 may include a coating, such as a lubricious coating for ease of insertion. The coating can be applied to solid or hollow microneedles and to any of the microneedle constructions disclosed herein. The coating may extend the full length of the microneedles 18 or only partially along the length. For example, the coating may be applied at the tip of the microneedles and extend for a short distance along the shaft of the microneedles 18. By employing a lubricious coating on the outer surface 38 of the microneedles 18, the insertion is easier thereby leading to the ability to provide more microneedles 10 in the array 10. This would lead to an increased dosage amount of the medicinal formulation provided in the microneedle array 10. Suitable lubricious coatings include, but are not limited, to polyvinylpyrrolidone (PVP), polyurethanes, polyacrylic acid, polyethylene oxide, polysaccharides, hydrophobic polymers such as polytetrafluoroethylene and silicone.

In other constructions, the microneedles 18 may include a coating with antimicrobial agents to allow for long term use of the array 10. Suitable antimicrobial agents include, but are not limited to Penicillins, Penicillin V, Penicillin G, Amoxicillin, Ampicillin, Cloxacillin, Methicillin, Amoxicillin+Clavulanate (Augmentin), Ticarcillin+Clavulanate, Nafcillin, 1st Generation Cephalosporins, Cephalexin (Keflex), Cefazolin, Cefadroxil, (LEXie DROpped ZOLa), 2nd Generation Cephalosporins, Ceflaclor, Cefuroxime, (LACking URine), 3rd Generation Cephalosporins, Cefotaxime, Cefoperazone, Cephtriaxone, 4th Generation Cephalosporins, Cefepime, Tetracyclines, Tetracycline, Minocycline, Doxycycline, Macrolides, Azithromycin, Erithromycin, Clarithromycin, Lincosamides/Lincosamines, Clindamycin (Cleocin), Sulfonamides/Sulfa Drugs, Sulfamethoxazole-Trimethoprim (generic), (Bactrim), (Cotrim), (Septra), Fluoroquinolones, Ciprofloxacin (Cipro), Norfloxacin, Ofloxacin, Levofloxacin, Aminoglycosides, Streptomycin, Tobramycin, Gentamycin, Amikacin.

In fabricating the microneedle array 10, the coating may be applied by dip coating or spray coating onto the microneedles 18. Alternatively, in a molding process of the microneedle array 10, the coating could be added to the mold such that an outer layer of the microneedles 18 contain the coating upon completion of the fabrication process.

In conventional microneedle arrays with microneedles fabricated as dissolvable or biodegradable, the microneedles are comprised of a non-resorbable metal or a resorbable or non-resorbable polymer material. In contrast, embodiments of the invention include microneedles comprising a bioresorbable metal, such as magnesium, zinc, iron, tungsten, molybdenum, silver, gold, platinum, alloys thereof, other water soluble metals, heir alloy, and combinations thereof. Magnesium employed as the microneedle material would dissolve after application and release the medicinal formulation into the bod to elicit its biological response. A resorbable metal microneedle array offers an advantage of allowing a more structurally stable configuration that would enable the formation of a hollow resorbable needle.

Various features and advantages of the invention are set forth in the following claims.

What is claimed is:

1. A microneedle array comprising:
a base plate;
a plurality of microneedles extending from the base plate;
wherein at least one of the microneedles includes an anchoring system configured to retain the array in position on skin for a treatment period.

2. The microneedle array of claim 1, wherein the anchoring system comprises a plurality of protrusions extending radially outward from an outer surface of the microneedle.

3. The microneedle array of claim 2, wherein the protrusions extend perpendicularly relative to a longitudinal axis of the microneedle.

4. The microneedle array of claim 1, wherein the anchoring system comprises a plurality of barbs extending outward from an outer surface of the microneedle at an angle relative to a longitudinal axis of the microneedle.

5. The microneedle array of claim 4, wherein the plurality of barbs are angled in a direction toward a tip of the microneedle.

6. The microneedle array of claim 1, wherein the anchoring system comprises a plurality of grooves formed within an outer surface of the microneedle and extending parallel to a longitudinal axis of the microneedle.

7. The microneedle array of claim 1, wherein the anchoring system comprises a groove formed within an outer surface of the microneedle and spiraling around the outer surface of the microneedle.

8. The microneedle array of claim 1, wherein the anchoring system comprises a step extending radially inward from an outer surface of the microneedle.

9. The microneedle array of claim 8, wherein the step extends perpendicularly related to a longitudinal axis of the microneedle.

10. The microneedle array of claim 1, wherein the anchoring system comprises a swellable portion activated upon contact with bodily fluid.

11. The microneedle array of claim 10, wherein the swellable portion is at a tip of the microneedle.

12. The microneedle array of claim 1, wherein the anchoring system comprises a swellable portion activated based upon a temperature threshold.

13. A microneedle array comprising:
a base plate; and
a plurality of microneedles extending from the base plate;
wherein at least one of the microneedles comprises a bioresorbable material.

14. The microneedle array of claim 13, wherein the bioresorbable material comprises a bioresorbable polymer, bioresorbable metal, or a combination thereof.

15. The microneedle array of claim 14, wherein the bioresorbable metal comprises magnesium, zinc, iron, tungsten, molybdenum, silver, gold, platinum, an alloy thereof, a water soluble metal, heir alloy or a combination thereof.

16. A microneedle array comprising:
a base plate; and
a plurality of microneedles extending from the base plate;
wherein at least one of the microneedles includes a lubricious coating.

17. The microneedle array of claim 15, wherein the lubricious coating comprises polyvinylpyrrolidone (PVP), a polyurethane, a polyacrylic acid, polyethylene oxide, a polysaccharide, polytetrafluoroethylene, silicone or a combination thereof.
